(19) United States
(12) Patent Application Publication
(10) Pub. No.: US 2009/0234224 A1
(43) Pub. Date: Sep. 17, 2009

(54) Positioning Device and a System for Detecting the Position of Such a Device

(75) Inventors: Roman Iustin, Molndal (SE); Tomas Gustafsson, Molndal (SE); Bo Lennernas, Uddevalla (SE); Bengt Rosengren, Hovas (SE)

Correspondence Address: BIRCH STEWART KOLASCH & BIRCH, PO BOX 747, FALLS CHURCH, VA, 22040-0747, US

(73) Assignee: MICROPOS MEDICAL AB, Goteborg (SE)

(21) Appl. No.: 12/083,515

(22) PCT Filed: Oct. 5, 2006
(86) PCT No.: PCT/SE2006/001135

§ 371 Date: Apr. 11, 2008

(30) Foreign Application Priority Data
Oct. 14, 2005 (SE) 0502273-6

(51) Int. Cl. A61B 5/05

(52) U.S. Cl. 600/424

(57) ABSTRACT

The present invention relates to a positioning device 10, 20, 30, 40, 50 arranged in relation to a target area inside a body. The device comprises at least one transmitter 11, 21-22, 32, 41, 51, and each transmitter is arranged to emit an electromagnetic signal. The electromagnetic signal is adapted to propagate with a wavelength in the body so that a phase difference and/or an amplitude difference of said electromagnetic signal in at least three positions 18 is detectable by a receiver 19 for tracking variations of a position of the positioning device relative to the receiver. The wavelength is selected so that a distance from the transmitter to each of the three positions is within the same integer number of wavelengths of the electromagnetic signal. Each transmitter is connectable to an externally arranged control unit 15, where said electromagnetic signal of each transmitter is generated before transmission. Each transmitter and receiver preferably operates in a near field region. The invention also relates to a system for tracking the position of a target area using a positioning device.

### TECHNICAL FIELD

The present invention relates to a positioning device configured to be arranged to a target area inside a body. The invention also relates to a system for detecting a position of such a device.

### BACKGROUND TO THE INVENTION

In the published Swedish patent SE 511 291 is a method disclosed for administrating treatment radiation towards a target area, such as a tumour, within a patient. The administration direction is determined by providing a reference object which is inserted into the body of the patient close to the target area. The reference object has reference markings that may be detected using computed tomography (CT) or X-ray.

A drawback with the prior art is that an amount of radiation has to be used to detect the position of the reference object. The patient is submitted to an unnecessary exposure of radiation before the actual treatment may commence.

A solution to this drawback is directed to the use of magnetically activated sensors, or markers. These may be wirelessly implanted into a body and the markers could be tracked using an induced magnetic field. The sensors are normally permanently inserted into the body, and may migrate over time.

A drawback with this solution is that Magnetic Resonance Imaging (MRI) may not be used unless the sensors are removed.

Another solution is to provide a marker with an electromagnetic transmitter at the tip of the device that is inserted into the body and a receiver unit is placed outside the body to track the movement of the tip during the inserting procedure.

This solution has a drawback in that it experience a limited resolution, which leads to that x-ray normally still needs to be used to verify the position of the sensor/marker.

### SUMMARY OF THE INVENTION

An object with the invention is to provide a device which may be used in a system to detect the position of the device which overcomes the above mentioned drawbacks.

The object is achieved in a device configured to be arranged in relation to a target area within a body. The device comprises at least one transmitter configured to emit an electromagnetic signal, which signal is received in at least three positions by a receiver arranged outside the body. The electromagnetic signal is generated in an externally arranged control unit and the device is configured to be connected to the control unit through transmission lines. The frequency of the emitted electromagnetic signal must be selected in such a way that a distance from each transmitter to each of the receiver's positions is within the same integer number of wavelengths of the electromagnetic signal.

The object is also achieved by a system using a device as described above.

An advantage with the present invention is that x-ray is not needed to be used to verify the position of the device.

Another advantage is that the present invention can be used to locate the device at all times after the device has been inserted into a body, which makes it possible to verify its position inside the body during e.g. treatment or feeding.

Still another advantage with the present invention is that there is a possibility for automation, i.e. radiation treatment can be administered automatically.

Still another advantage with the present invention is that the need for picture estimation by a doctor is eliminated, due to auto-positioning of the device.

An advantage with a preferred embodiment is that MRI may be used on the body since the positioning device easily may be removed from the body without the need for a surgical procedure.

### BRIEF DESCRIPTION OF THE DRAWINGS

FIG. 1 shows a first embodiment of a positioning device according to the invention.

FIG. 2 shows a second embodiment of a positioning device according to the invention.

FIG. 3 shows a third embodiment of a positioning device according to the invention.

FIG. 4 shows a fourth embodiment of a positioning device according to the invention.

FIG. 5 shows a plot illustrating the near field effect of electromagnetic signals.

FIG. 6 shows a fifth embodiment of a positioning device according to the invention.

### DETAILED DESCRIPTION OF THE PREFERRED EMBODIMENTS

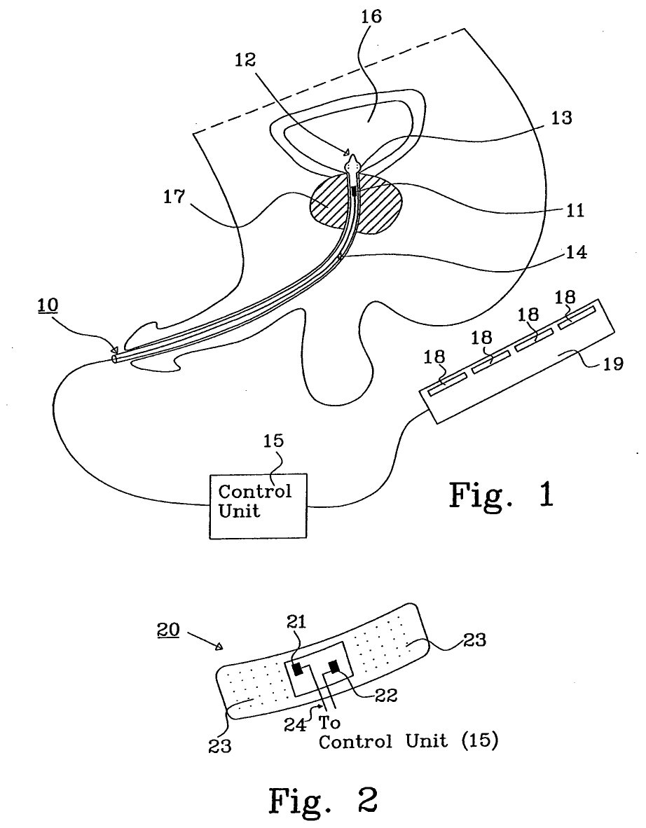

FIG. 1 shows a first embodiment of a positioning device, which in this embodiment is a removable catheter 10, according to the invention. The catheter 10 comprises a transmitter 11 arranged close to the tip 12 of the catheter. An expandable portion 13 of the catheter 10 is provided between the transmitter 11 and the tip 12 of the catheter 10. A transmission line 14 connects the transmitter 11 with an externally arranged control unit 15.

This type of positioning device is preferably used during cancer treatment, such as prostate cancer, breast cancer uterus cancer, and a very accurate position of the prostate 17 (target area) is required to optimise the treatment procedure. The catheter is in this example inserted through a natural opening, as illustrated in FIG. 1, and the expandable portion 13 is inserted into the bladder 16 where it is expanded to fixate the transmitter 11 relative to the prostate 17 before the treatment begins.

An electromagnetic signal is generated in the control unit 15 and is thereafter transmitted from the transmitter 11. The electromagnetic signal is adapted to propagate with a wavelength in the body and, in a first example, a phase difference of the electromagnetic signal is detected in at least three positions 18, preferably four or more positions, by a receiver 19 arranged outside the body. The wavelength is selected so that a distance from the transmitter 11 to each of said at least three positions 18 is within the same integer number of wavelengths of the electromagnetic signal. The distance between each transmitter and the positions 18 of the receiver 19 is preferably selected so that they operate in a near field region. A prior art detector system is described in an international patent application PCT/SE05/000646, assigned to the same applicant, wherein a transmitter arranged in relation to a target area inside a body transmits a signal having a frequency within the range of 5-350 MHz and a phase difference from the transmitted signal is detected by a receiver at three, or more, positions to track variations in position of the transmitter.

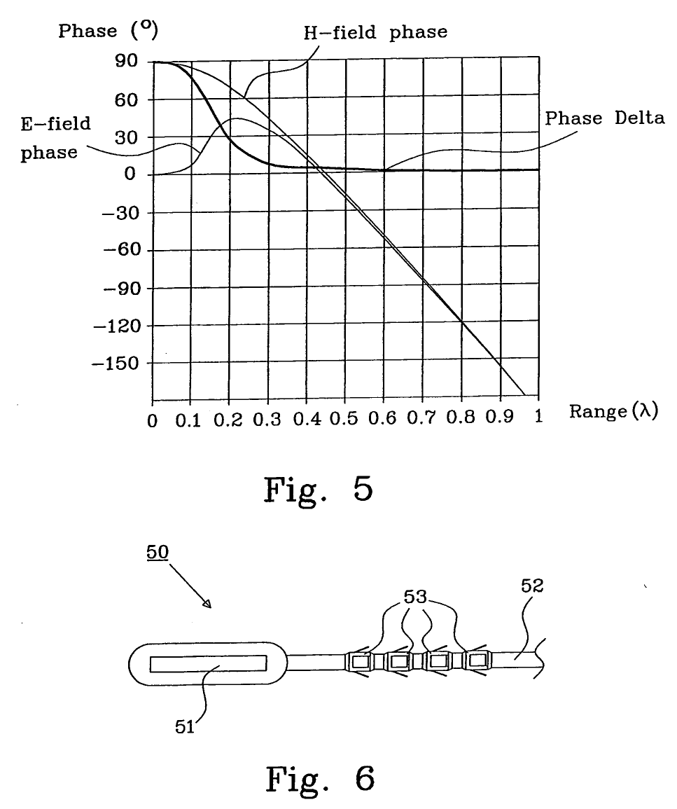

The prior art system described defines a transmitter and a multiple of receiving antennas that operates in a near field region. The behaviour of an electromagnetic signal in the near field region is known for a skilled person and is described in a publication with the title “Near field Phase Behavior”, by Hans Gregory Schantz, IEEE APS Conference July 2005. In this publication the author presents a reprint of a plot published in “Electric waves”, by Heinrich Hertz, London, Macmillian & Co. 1893, page 152 and a plot, shown in FIG. 5, was published by Q-track in 2004.

The plot describes the phase behaviour of the magnetic field (H-field) and the electrostatic filed (E-field) below one wavelength of an electromagnetic signal. In this near field region of an antenna, the magnetic field and electrostatic field phases radically diverge, and in a far field region, many wavelengths away from a transmit antenna, the magnetic and electrostatic field move with perfect synchronized phase. FIG. 5 illustrates the effect of the near field region, and the phase delta between the magnetic field and electrostatic field at zero λ is 90 degrees, which decreases to a phase difference of 0 degrees at one λ.

The separation of the magnetic field and the electrostatic field in the near field region opens up a number of possibilities to construct improved measurement systems. The shape of the wave front of the electromagnetic signal may be used to determine the distance between the transmitter and the receiver. It is also advantageous to increase the sensitivity of the measurement system by introducing electrostatically shielded antennas, which is possible since the magnetic field and electrostatic field are separated in the near field region, whereby the magnetic field is used to determine the variations of the position of the transmitter.

A more detailed description of the detector system may be found in the international patent application with the application number PCT/SE05/000646, which is hereby incorporated by reference.

In a second example, amplitude difference of the electromagnetic signal is detected instead of the phase difference as described above. A transmitter arranged in relation to a target area inside a body transmits a signal having a frequency within the range of 1 kHz-350 MHz and an amplitude difference from the transmitted signal is detected by a receiver at three, or more, positions to track variations in position of the transmitter. The amplitude of the magnetic field is preferably measured when operating in near field, for instance by measuring absolute value or mean value of the magnetic field.

It is of course possible to combine the above described examples and use both phase and amplitude difference to determine variations of the position of the transmitter.

The receiver 19, comprising a multiple of antennas at separate positions 18 (in this example four positions), is positioned on the outside of the body and is also connected to the control unit 15, and a very accurate tracking of the transmitter 11, and thus the prostate 17, may be performed. When radiotherapy treatment is completed, the removable catheter 10 is removed by deflating the expandable portion 13 and withdrawing the catheter 10.

FIG. 2 shows a second embodiment of a positioning device, which in this embodiment is a plaster 20, which easily may be attached to the skin of a patient in an appropriate position. The plaster 20 comprises in this embodiment two transmitters 21 and 22 in a central part of the plaster 20 and two adhesive portions 23 to securely attach the plaster to the skin. The transmitter 21, 22 are connectable to a Control Unit, similar to the one described in connection with FIG. 1, through transmission lines 24.

The plaster could, for instance, be used when a patient having breast cancer is exposed to radiotherapy treatment. The plaster 20 is then attached to the breast in such a way that a relative distance between a target area in the breast and each transmitter 21 and 22 is established. The breathing of the patient, and thus the movement of the breast due to breathing, can be tracked, and the radiotherapy treatment can now be controlled more accurately by adapting the exposure dose to the position of the target area. It is of course possible to have only one transmitter, or even have more than two transmitters, without deviating from the scope of the invention as defined in the claims.

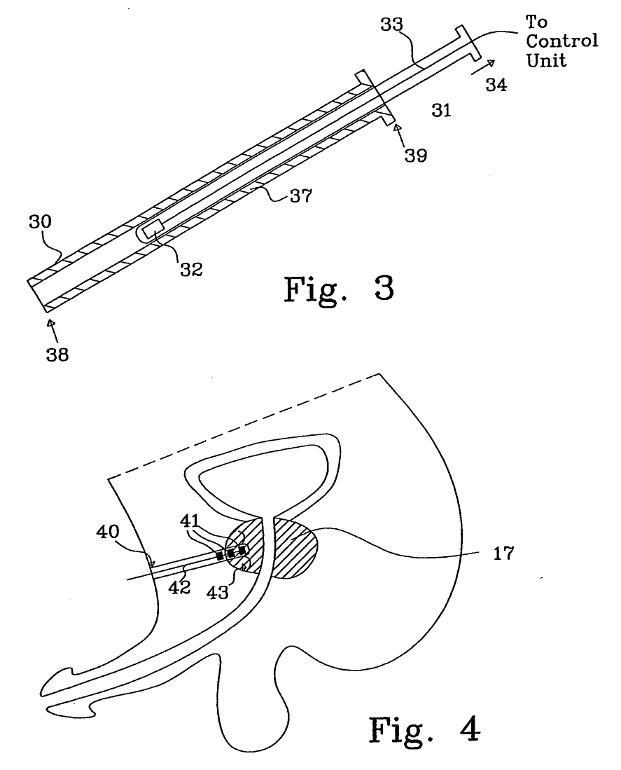

FIG. 3 shows a third embodiment of a positioning device, in which in this embodiment is a feeding tube 30 having a first end 38 being arranged to be inserted into the stomach through the nose of a patient, and a second end 39 being arranged to be connected to a feeding arrangement (not shown). It is essential that the feeding tube 30 is not misdirected during the inserting procedure and by mistake entered into one of the lungs. The feeding tube 30 is therefore provided with a removable insert 31 having a transmitter 32 arranged in the end of the insert 31 that is intended to be aligned with the first end 38 of the feeding tube 30 during the inserting procedure. The transmitter 32 is provided with a transmission line 33, which is connectable to a control unit (not shown) of the same type as described in connection with FIG. 1.

When the feeding tube has been placed in the right position, the insert 31 is withdrawn from the feeding tube 30 as illustrated by the arrow 34 in FIG. 3. The feeding through the feeding tube 30 may thereafter commence.

In an alternative embodiment, the transmitter 32 and the transmission line 33 are integrated into a wall 37 of the feeding tube 30, which has the advantage that a removable insert is not necessary. The position of a hollow position secured device, such as a feeding tube, can continuously be monitored to track the position of the feeding tube 30 during feeding.

FIG. 4 shows a fourth embodiment of a positioning device, which in this embodiment is a catheter 40, according to the present invention. The catheter 40 is kept in place at least during the radiotherapy treatment and could thereafter be removed or left in place. The catheter 40 is in this embodiment provided with three transmitters 41 having transmission lines 42 that are connectable to a control unit (not shown). The catheter is further provided with fastening means 43 in the shape of hooks, or other forms, e.g. spiral, that securely attaches the catheter 40 with the transmitters 41 to the prostate 17.

A wire-based implant covered with biocompatible material, i.e. without the need of a protective catheter, is also conceivable.

FIG. 6 shows a fifth embodiment of a positioning device 50, similar to the positioning device described in connection with FIG. 4. No catheter is provided and one or more transmitters 51 are arranged in a biocompatible capsule, and transmission lines connecting the transmitters 51 with an external control unit (not shown) are contained within a biocompatible lead 52. In this embodiment, the fastening means are implemented as a multiple of tine elements 53, in this example four elements, arranged on the outside of the lead 52. Further examples of tine elements may be found in the published US application US 2006/0129218, assigned to Medtronic, Inc.

1-17. (canceled)

18. A device configured to be arranged in relation to a target area inside a body, said device comprising at least one transmitter, each transmitter is configured to emit an electromagnetic signal, wherein said electromagnetic signal is adapted to propagate with a wavelength in said body so that said electromagnetic signal in at least three positions is detectable by a receiver for tracking variations of a position of each transmitter relative to said receiver, wherein said wavelength is selected so that a distance from each transmitter to each of said at least three positions is within the same integer number of wavelengths of the electromagnetic signal, said transmitter is connectable to an externally arranged control unit in which said electromagnetic signal of each transmitter is generated before transmission, and each transmitter and receiver operates in a near field region.

19. The device according to claim 18, wherein each transmitter is configured to emit an electromagnetic signal that is adapted to propagate with a wavelength in said body so that a phase difference of said electromagnetic signal in at least three positions is detectable by the receiver for tracking variations of the position of each transmitter relative to said receiver.

20. The device according to claim 18, wherein each transmitter is configured to emit an electromagnetic signal that is adapted to propagate with a wavelength in said body so that an amplitude difference of said electromagnetic signal in at least three positions is detectable by the receiver for tracking variations of the position of each transmitter relative to said receiver.

21. The device according to claim 18, wherein said transmitter is connectable to said externally arranged control unit via transmission lines.

22. The device according to claim 18, wherein said device is provided with a means to secure said at least one transmitter relative to the target area.

23. The device according to claim 22, wherein an expanding portion of the device is used to secure the transmitter.

24. The device according to claim 22, wherein said device further comprises at least one adhesive portion to secure the transmitter to a surface of the body, whereby a relative distance to the target area is established.

25. The device according to claim 22, wherein said device comprises extractable hooks to secure the transmitter.

26. The device according to claim 5, wherein said device comprises a multiple of tine elements to secure the transmitter.

27. The device according to claim 18, wherein said device further comprises a tube arranged to be inserted into a natural opening of the body.

28. The device according to claim 27, wherein said tube is provided with a wall and said at least one transmitter is integrated in the wall.

29. The device according to claim 27, wherein said at least one transmitter is removably arranged within said tube.

30. A device configured to be arranged in relation to a target area inside a body, said device comprising at least one transmitter, each transmitter is configured to emit an electromagnetic signal, wherein said electromagnetic signal is adapted to propagate with a wavelength in said body so that a phase difference of said electromagnetic signal in at least three positions is detectable by a receiver for tracking variations of a position of each transmitter relative to said receiver, wherein said wavelength is selected so that a distance from each transmitter to each of said at least three positions is within the same integer number of wavelengths of the electromagnetic signal, said transmitter is connectable to an externally arranged control unit in which said electromagnetic signal of each transmitter is generated before transmission.

31. The device according to claim 30, wherein said transmitter is connectable to said externally arranged control unit via transmission lines.

32. The device according to claim 30, wherein said device is provided with a means to secure said at least one transmitter relative to the target area.

33. The device according to claim 32, wherein an expanding portion of the device is used to secure the transmitter.

34. The device according to claim 32, wherein said device further comprises at least one adhesive portion to secure the transmitter to a surface of the body, whereby a relative distance to the target area is established.

35. The device according to claim 32, wherein said device comprises extractable hooks to secure the transmitter.

36. The device according to claim 32, wherein said device comprises a multiple of tine elements to secure the transmitter.

37. The device according to claim 30, wherein said device further comprises a tube arranged to be inserted into a natural opening of the body.

38. The device according to claim 37, wherein said tube is provided with a wall and said at least one transmitter is integrated in the wall.

39. The device according to claim 37, wherein said at least one transmitter is removably arranged within said tube.

40. A system for tracking a position of a target area inside a body, said system comprising:
a positioning device comprising at least one transmitter arranged in relation to said target area, each transmitter emitting an electromagnetic signal, wherein said electromagnetic signal is adapted to propagate with a wavelength in said body so that said electromagnetic signal in at least three positions is detectable by a receiver for tracking variations of a position of each transmitter relative to said receiver, wherein said wavelength is selected so that a distance from the transmitter to each of said at least three positions is within the same integer number of wavelengths of the electromagnetic signal, the system further comprising:
an externally arranged control unit, wherein each transmitter is connected to said control unit, in which said electromagnetic signal of each transmitter is generated before transmission, and
each transmitter and receiver are arranged to operate in a near field region.

41. The system according to claim 40, wherein each transmitter is configured to emit an electromagnetic signal that is adapted to propagate with a wavelength in said body so that a phase difference of said electromagnetic signal in at least three positions is detectable by the receiver for tracking variations of the position of each transmitter relative to said receiver.

42. The system according to claim 40, wherein each transmitter is configured to emit an electromagnetic signal that is adapted to propagate with a wavelength in said body so that an amplitude difference of said electromagnetic signal in at least three positions is detectable by the receiver for tracking variations of the position of each transmitter relative to said receiver.

43. A system for tracking a position of a target area inside a body, said system comprising:
a positioning device comprising at least one transmitter arranged in relation to said target area, each transmitter emitting an electromagnetic signal, wherein said electromagnetic signal is adapted to propagate with a wavelength in said body so that a phase difference of said electromagnetic signal in at least three positions is detectable by a receiver for tracking variations of a position of each transmitter relative to said receiver, wherein said wavelength is selected so that a distance from the transmitter to each of said at least three positions is within the same integer number of wavelengths of the electromagnetic signal, characterized in that the system further comprising:
an externally arranged control unit, wherein each transmitter is connected to said control unit, in which said electromagnetic signal of each transmitter is generated before transmission.
